(19) United States
(12) Patent Application Publication
(10) Pub. No.: US 2024/0020782 A1
(43) Pub. Date: Jan. 18, 2024

(54) METHOD AND SYSTEM FOR REDUCING GREENHOUSE GAS USING LIVETOCK GENOMIC INFORMATION

(71) Applicants: INDUSTRIAL COOPERATION FOUNDATION CHONBUK NATIONAL UNIVERSITY, Jeonju-si (KR); MELLIENS INC., Jeonju-si (KR)

(72) Inventors: Hak Kyo LEE, Jeonju-si (KR); Jae Young HEO, Jeonju-si (KR); Ki Duk SONG, Jeonju-si (KR); Dong Hyun SHIN, Jeonju-si (KR); Jin Weon YOON, Jeonju-si (KR)

(73) Assignees: INDUSTRIAL COOPERATION FOUNDATION CHONBUK NATIONAL UNIVERSITY, Jeonju-si (KR); MELLIENS INC., Jeonju-si (KR)

(21) Appl. No.: 17/607,077

(22) PCT Filed: Sep. 14, 2021
(86) PCT No.: PCT/KR2021/012456

§ 371 Date: Oct. 28, 2021

(30) Foreign Application Priority Data
Jul. 21, 2021 (KR) 10-2021-0095839

(51) Int. Cl. G06Q 50/26; G06Q 40/04

(52) U.S. Cl. CPC G06Q 50/26; G06Q 40/04

(57) ABSTRACT

A method and system for reducing greenhouse gas using livestock genomic information is disclosed. The method is a method for molecular breeding for reducing carbon footprint for group subjects, including obtaining genomic information for each subject in a reference group; estimating a breeding value of a carbon emission-related trait for each subject based on the genomic information, after setting a meat production amount that is inversely proportional to the amount of carbon emission per weight as the carbon emission-related trait of each subject; and selecting subjects having a breeding value corresponding to a reference ratio or higher among the breeding values as subjects to be crossed

### CROSS-REFERENCE TO RELATED APPLICATION

This application claims priority to and the benefit of Korean Patent Application No. 10-2021-0095839, filed on Jul. 21, 2021, the disclosure of which is incorporated herein by reference in its entirety.

### BACKGROUND

### Field of the Disclosure

The present invention relates to a technique for reducing greenhouse gas, and more specifically to a method and system for reducing carbon footprint using livestock genomic big data.

### Description of Related Art

Greenhouse gas emissions, which are rapidly increasing along with industrial development, have a significant impact on climate change. Accordingly, countries around the world are making efforts to reduce greenhouse gas emissions. That is, by signing climate agreements, countries around the world are aiming to of reduce 50% of carbon emissions by 2030.

For example, the global anthropogenic carbon emissions amount to more than 50 billion tons per year (equivalent to carbon dioxide), of which the carbon emissions of the livestock sector account for about 16.5%. In addition, the meat-related sector accounts for more than 61% of carbon emissions in the livestock sector (FAQ, 2017). The reason why the share of carbon emissions in the livestock sector is large is that methane, which has a high greenhouse gas effect (about 28 times that of carbon dioxide), is generated during the intestinal fermentation, excretion and manure treatment of livestock. For example, the amount of carbon emitted by two cows is equivalent to that of one vehicle.

Meanwhile, the price of carbon credits is increasing every year. In particular, the global livestock sector's carbon emissions amount to about 8 billion tons per year. Reducing this by 50% can reduce carbon emissions by about 4 billion tons per year, which can contribute to the development of a low-carbon economy by reducing climate change and forming a new market for carbon credits. For example, when reducing carbon emissions by 4 billion tons in the livestock sector, as of 2021, about $200 billion (220 trillion won) of carbon credits per year will be formed, creating new jobs related to the carbon economy and building a low-carbon industrial ecosystem.

As part of an effort to reduce carbon emissions in the livestock sector, there is related art such as KR10-2014-0055882 A for reducing methane emissions of livestock through a specific feed. However, in the case of this related art, carbon emission reduction can be calculated only based on the amount of raw and subsidiary materials such as feed and the like which are input in the production process of livestock, and thus, there is a problem in that it cannot provide a technique for reducing carbon footprint.

In particular, the situation is that a technique that can predict the carbon emissions of livestock and their offspring and reduce the carbon print based thereon has not yet been developed.

### SUMMARY OF THE INVENTION

In order to solve the problems of the prior art as described above, it is an object of the present invention to predict the amount of carbon emissions for each subject using livestock genomic big data, and to provide a technique for reducing the carbon footprint of the corresponding livestock based thereon.

However, the problems to be solved by the present invention are not limited to the above-mentioned problem, and other problems not mentioned can be clearly understood by those of ordinary skill in the art to which the present invention pertains from the description below.

In order to solve the above-described problem, the method according to an exemplary embodiment of the present invention is a method for molecular breeding for reducing carbon footprint for group subjects, including obtaining genomic information for each subject in a reference group; estimating a breeding value of a carbon emission-related trait for each subject based on the genomic information, after setting a meat production amount that is inversely proportional to the amount of carbon emission per weight as the carbon emission-related trait of each subject; and selecting subjects having a breeding value corresponding to a reference ratio or higher among the breeding values as subjects to be crossed.

The method according to an exemplary embodiment of the present invention may further include calculating reference carbon emission information, which is information related to the current average carbon emission of each subject, using the breeding value of each subject, and calculating target carbon emission information, which is information related to the estimated average carbon emission amount of each offspring subject of the subjects to be crossed, using the breeding values of the subjects to be crossed; and using by comparing the reference carbon emission information and the target carbon emission information.

The using may include calculating the estimated carbon reduction amount based on the target carbon emission information and the reference carbon emission information to use the estimated carbon reduction amount.

The using may include performing futures trading for carbon credit trading based on the estimated carbon reduction amount.

The estimating may include estimating the breeding value using the following formula:

Y=Xb+Zu+e

(where Y is the amount of meat production per subject, X is environmental effect information, Z is genomic information, b is a fixed effect vector, u is a breeding value vector, and e is an error vector).

The environmental effect information may be at least one selected from birth date information, slaughter date information, slaughter age information, farm information, slaughterhouse information and gender information.

The method according to another exemplary embodiment of the present invention is a method performed in a system for providing information for reducing carbon footprint for group subjects, including storing genomic information for each subject in a reference group; estimating a breeding value of a carbon emission-related trait for each subject based on the genomic information, after setting a meat production amount that is inversely proportional to the amount of carbon emission per weight as the carbon emission-related trait of each subject; selecting subjects having a breeding value corresponding to a reference ratio or higher among the breeding values as subjects to be crossed; and displaying processed information or transmitting the same to another device or system.

The processed information may include at least one of information on the breeding value and information on the subjects to be crossed.

The method according to another exemplary embodiment of the present invention may further include calculating reference carbon emission information, which is information related to the current average carbon emission of each subject, using the breeding value of each subject, and calculating target carbon emission information, which is information related to the estimated average carbon emission amount of each offspring subject of the subjects to be crossed, using the breeding values of the subjects to be crossed; and comparing the reference carbon emission information and the target carbon emission information,

The processed information may include at least one of information on the breeding value, information on the subjects to be crossed, the reference carbon emission information, the target carbon emission information and result information from the comparison.

The comparing may include calculating the estimated carbon reduction amount based on the target carbon emission information and the reference carbon emission information.

The result information from the comparison may include information on the estimated carbon reduction amount.

The method may further include performing futures trading for carbon credit trading by accessing a trading system for trading carbon credits based on the information on the estimated carbon reduction amount.

The system according to an exemplary embodiment of the present invention is a system for providing information for reducing carbon footprint for group subjects, including a memory for storing genomic information for each subject in a reference group; and a controller for processing by using the stored information,

The controller may estimate a breeding value of a carbon emission-related trait for each subject based on the genomic information, after setting a meat production amount that is inversely proportional to the amount of carbon emission per weight as the carbon emission-related trait of each subject, and select subjects having a breeding value corresponding to a reference ratio or higher among the breeding values as subjects to be crossed.

The controller may display processed information on a display or transmits the same to another device of system, and the processed information imay include at least one of information on the breeding value and information on the subjects to be crossed.

The controller may calculate reference carbon emission information, which is information related to the current average carbon emission of each subject, using the breeding value of each subject, calculate target carbon emission information, which is information related to the estimated average carbon emission amount of each offspring subject of the subjects to be crossed, using the breeding values of the subjects to be crossed, and compare the reference carbon emission information and the target carbon emission information, and the processed information may include at least one of information on the breeding value, information on the subjects to be crossed, the reference carbon emission information, the target carbon emission information and result information from the comparison.

The controller may calculate the estimated carbon reduction amount based on the target carbon emission information and the reference carbon emission information upon the comparison, and the result information from the comparison may include information on the estimated carbon reduction amount.

The controller may perform futures trading for carbon credit trading by accessing a trading system for trading carbon credits based on the information on the estimated carbon reduction amount.

The present invention configured as described above proposes a new technique that can specifically derive the predicted carbon reduction amount for offspring subjects (future generation) selected based on the genomic information of the target subjects (current generation), and accordingly, it has an advantage of providing a variety of information related to reducing carbon footprint, molecular breeding and the like.

In addition, since the present invention can provide a technique that can calculate and use information on various carbon emissions based on the estimated breeding value by estimating the breeding value of carbon emission-related traits using genomic information, it has an advantage of contributing to greenhouse gas reduction in the livestock sector.

However, the effects obtainable in the present invention are not limited to the above-mentioned effects, and other effects not mentioned will be clearly understood by those of ordinary skill in the art to which the present invention pertains from the following description.

### BRIEF DESCRIPTION OF THE DRAWINGS

FIG. 1 shows a block configurational diagram of a system 100 according to an exemplary embodiment of the present invention.

FIG. 2 shows an example of improving carbon reduction efficiency (based on meat production efficiency) through the input of molecular breeding technology.

FIG. 3 shows an example of the improvement of carbon emission reduction efficiency based on molecular breeding technology by meat quality grade (based on meat production efficiency).

FIG. 4 shows an example of improving carbon reduction efficiency (based on carbon emissions) through the input of molecular breeding technology.

FIG. 5 shows an example of the improvement of carbon reduction efficiency (based on carbon emissions) based on molecular breeding technology for each meat quality grade.

FIG. 6 shows the effect of reducing carbon emissions per meat weight according to the prediction accuracy.

FIG. 7 shows an example of the estimation of the mean of the 1++ grade carbon emission group from 2020 to 2025 by the accuracy of simulation-based breeding values.

FIG. 8 shows an example of the effect of improving the carbon accumulation efficiency (meat weight) of the improved group after applying the genetic ability prediction.

FIG. 9 shows an example of the effect of improving the carbon emission reduction efficiency of the improvement group when the genomic information utilization technology is applied.

FIG. 10 shows an example of the predicted value of the carbon emission reduction effect of the improvement group (based on the annual production of Korean beef steer) through the application of the genetic ability prediction platform.

FIG. 11 shows the appearance rate by breed, gender, meat quality and meat quantity grades from 2017 to 2019.

FIG. 12 shows a flowchart of the method according to various exemplary embodiments of the present invention.

FIG. 13 is a block configurational diagram of a comprehensive system 1 according to an exemplary embodiment of the present invention.

FIGS. 14 and 15 show various examples of electronic devices connecting to the comprehensive system 1 according to an exemplary embodiment of the present invention.

### DETAILED DESCRIPTION OF THE INVENTION

The above Objects and means of the present invention and the effects thereof will become more clear through the following detailed description in relation to the accompanying drawings, and accordingly, those of ordinary skill in the art to which the present invention pertains can easily practice the technical idea of the present invention. In addition, in the description of the present invention, if it is determined that a detailed description of the known technology related to the present invention may unnecessarily obscure the gist of the present invention, the detailed description thereof will be omitted.

Unless otherwise defined, all terms used herein may be used with meanings commonly understood by those of ordinary skill in the art to which the present invention pertains. In addition, terms defined in a commonly used dictionary are not to be interpreted ideally or excessively unless specifically defined explicitly.

Hereinafter, a preferred exemplary embodiment according to the present invention will be described in detail with reference to the accompanying drawings.

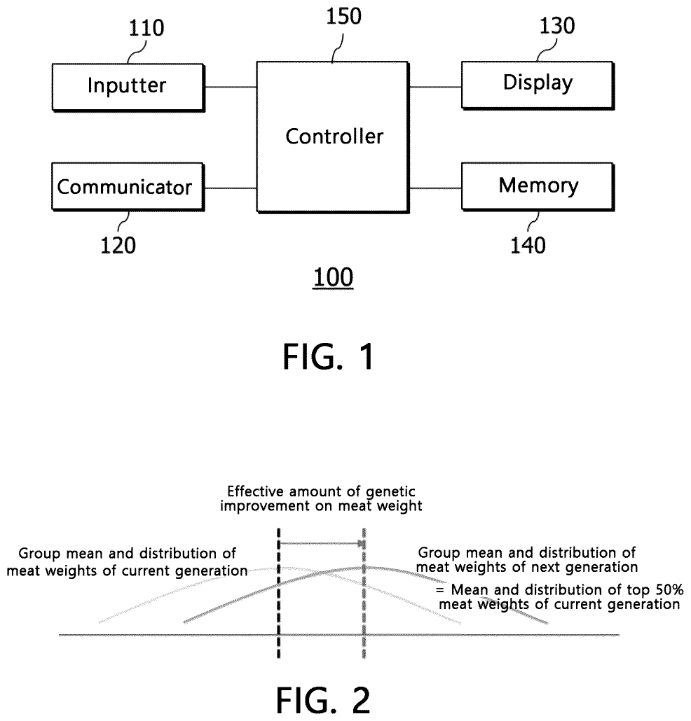

FIG. 1 shows a block configurational diagram of a system 100 according to an exemplary embodiment of the present invention.

In the conventional case, information on the average carbon emission of beef by country could be known, but the amount of carbon emission by subjects could not be known. In particular, based on genomic information, after deriving information related to individual carbon emissions of the current group subjects and information related to the estimated carbon emission for future offsprings of the breeding target subject selected from the group subjects, there is currently no technique capable of reducing the carbon footprint by using the derived information, and in order to solve the same, a system 100 according to the present invention is proposed.

That is, the system 100 is a system applicable to the livestock sector, and based on the genomic information of each livestock (subject) belonging to the current reference group, it provides various information for reducing the carbon footprint of the group subjects and molecular breeding accordingly.

In particular, the system 100 may estimate the breeding value of the carbon emission-related trait for each subject in the current reference population and future offspring based on the genomic information. In addition, information related to the current average carbon emission of each subject belonging to the current reference group (hereinafter, referred to as “reference carbon emission information”) and information related to the estimated average carbon emission of each offspring subject in the future (hereinafter, referred to as “target carbon emission information”) may be calculated.

In addition, the system 100 may provide various information by comparing the reference carbon emission information with the target carbon emission information. The system 100 may be connected to various electronic devices and the like through various wired/wireless communication methods.

The system 100 may be an electronic device capable of computing or may include a corresponding electronic device, and may operate as a server.

For example, the electronic device may be a desktop personal computer (PC), a laptop personal computer (PC), a tablet personal computer (PC), a netbook computer, a workstation, a personal digital assistant (PDA), a smartphone, a smartpad, a mobile phone or the like, but is not limited thereto.

Referring to FIG. 1, the system 100 may include an inputter 110, a communicator 120, a display 130, a memory 140, a controller 150 and the like.

The inputter 110 generates input data in response to various user inputs, and it may include various input means. For example, the inputter 110 may include a keyboard, a keypad, a dome switch, a touch panel, a touch key, a touch pad, and a mouse, a menu button and the like, but is not limited thereto.

The communicator 120 is a component that performs communication with another device (or system). For example, the communicator 120 may perform wireless communication such as 5^{th} generation communication (5G), long term evolution-advanced (LTE-A), long term evolution (LIE), Bluetooth, Bluetooth low energy (BLE), near field communication (NIT), Wi-Fi communication and the like, or perform wired communication such as cable communication and the like, but is not limited thereto.

The display 130 displays various image data on a screen and may be configured as a non-light-emitting panel or a light-emitting panel. For example, the display 130 may include a liquid crystal display (LCD), a light emitting diode (LED) display, an organic light emitting diode (OLED) display, a micro-electromechanical system (MEMS) display, an electronic paper display or the like, but is not limited thereto. In addition, the display 130 may be implemented as a touch screen or the like in combination with the inputter 110.

The memory 140 stores various types of information necessary for the operation of the system 100. For example, the stored information may include livestock information (including genomic information, etc.), processed information, program information related to a method to be described below and the like, but is not limited thereto. In this case, the processed information may include at least one of information on breeding values, information on subjects to be crossed, reference carbon emission information, target carbon emission information and result information for comparison.

For example, depending on the type, the memory 140 may include a hard disk type, a magnetic media type, a compact disc read-only memory (CD-ROM), an optical media type, a magneto-optical media type, a multimedia card micro type, a flash memory type, a read-only memory type, a random access memory type or the like, but is not limited thereto. In addition, the memory 140 may be a cache, a buffer, a main memory, an auxiliary memory or a separately provided storage system depending on the purpose/location thereof, but is not limited thereto.

The controller 150 may perform various control operations of the system 100. That is, the controller 150 may control the execution of a method to be described below by using the information stored in the memory 140, and may control the operations of the remaining components of the system 100, that is, the inputter 110, the communicator 120, the display 130, the memory 140 and the like. For example, the controller 150 may include a processor which is hardware, a process which is software that is executed in the corresponding processor and the like, but is not limited thereto.

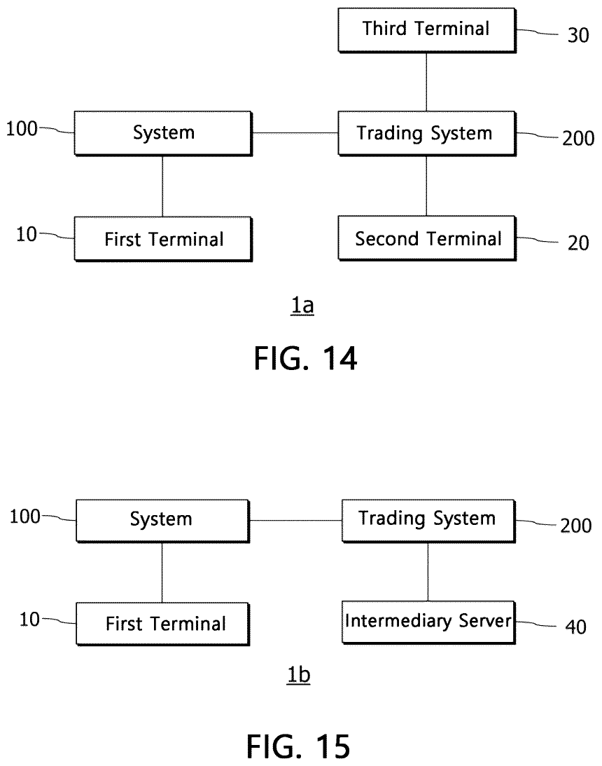

FIG. 13 is a block configurational diagram of a comprehensive system 1 according to an exemplary embodiment of the present invention. In addition, FIGS. 14 and 15 show various examples of electronic devices connecting to the comprehensive system 1 according to an exemplary embodiment of the present invention.

That is, the comprehensive system 1 is a system applicable to the livestock sector, and may include the above-described system 100 and a trading system 200 capable of performing carbon credit trading, respectively.

The technique according to this comprehensive system 1 may be applied from the livestock selection stage, for example, the parent (father or mother) livestock selection stage or the breeding start stage for young livestock. Referring to FIGS. 13 to 15, the comprehensive system 1 may further include various electronic devices connected to the system 100 and the trading system 200, that is, terminals 10, 20 and 30, an intermediary server 40 and the like. In this case, the system 100 and the trading system 200 or the systems 100 and 200 and each electronic device may be connected through various wired/wireless communication methods.

Meanwhile, the first terminal 10 is a terminal used by a collector to collect livestock information including genomic information, and is accessible to the system 100. That is, the collector may store the traceability information of livestock (livestock ID, date of birth, pedigree information, etc.), genome information according to the collection of a genome sample (hair root, blood, etc.) of the livestock in the first terminal 10. The first terminal 10 may transmit the stored information to the system 100.

However, the delivery of such livestock information may be performed through another terminal, server or the like that stores the corresponding information in addition to the first terminal 10. In addition, the livestock information is transmitted to the system 100 through a portable memory or the like in which it is stored, and may be pre-stored in the memory 140 of the system 100.

The system 100 stores and manages livestock information (including genomic information, etc.) on the target subject in the memory 140 (hereinafter, referred to as a “first function”). That is, the system 100 may store and manage information collected/transmitted through the first terminal 10 and the like. In addition, the system 100 may store and manage the information collected/transmitted through another server, a portable memory or the like.

Also, in addition to the first function, the system 100 may use livestock information (including genomic information, etc.) to provide various information for the reduction of carbon footprint and molecular breeding accordingly (hereinafter, referred to as a “second function”) based on the genomic information of each livestock (subject) belonging to the current reference group.

Meanwhile, the system 100 may transmit various information derived as a result of performing the second function to the trading system 200 that performs carbon trading and the like.

Meanwhile, the system 100 may include a first server performing a first function and a second server performing a second function. In this case, the first server and the second server are connected through wired/wireless communication. That is, the first server performs the first function according to the connection of the first terminal and the like, and transmits the stored livestock information to the second server. Afterwards, the second server performs the second function based on the transmitted livestock information, and transmits various information according to the execution of the second function to the trading system. In this case, the first server and the second server may each separately include an inputter 110, a communicator 120, a display 130, a memory 140, a controller 150 and the like.

Meanwhile, the trading system 200 is a system for performing carbon credit trading based on the carbon reduction amount of low-carbon certified livestock products. That is, if information on the predicted carbon reduction amount is received from the system 100, carbon credit trading may be performed based on the corresponding information. That is, it is possible to trade the carbon emission of the target carbon emission information, which has decreased compared to the reference carbon emission information, in the carbon credit market.

For example, the trading system 200 may carry out carbon credit trading based on the corresponding predicted carbon reduction amount, but may also perform futures trading. That is, predicted carbon emission that has decreased compared to a reference value may be futures traded in the carbon credit trading market.

Referring to FIG. 14, the second and third terminals 20 and 30 are electronic devices of the parties that access the trading system 200 and perform carbon credit trading. In this case, the second terminal 20 is a terminal used by a carbon credit seller. In other words, the seller refers to a person who sells carbon credits generated according to the predicted carbon reduction amount. In addition, the third terminal 30 may be a terminal used by a carbon credit trader buyer). In other words, buyers refer to those who purchase carbon credits generated according to the predicted carbon reduction amount.

In addition to the above, the second and third terminals 20 and 30 may be terminals used by a greenhouse gas emission certification institution, or terminals used by a person who checks whether low-carbon livestock is certified/carbon reduction amount.

However, as illustrated in FIG. 15, the trading system 200 may allow carbon credit trading to be made between the second and third terminals 20 and 30 through an intermediary server 40. In this case, the trading system 200 may transmit the information received from the system 100 to the intermediary server 40, and the intermediary server 40 may mediate the carbon credit trading according to the operation of the above-described trading system 200 based on the corresponding information.

Meanwhile, in relation to the trading system 200, the concept of carbon credit trading used in the present invention is as follows.

That is, the term ‘carbon emission right’ is used very commonly in Korea, but in the EU, etc., the quota (EUA) and credit (CER/ERO are relatively clearly distinguished and the corresponding ‘emission right’ is not used well.

The Kyoto Protocol, which formed the framework far the international response system for climate change, proposes the ‘Kyoto Flexible Mechanism’, a market-based mechanism, to alleviate the burden of greenhouse gas reduction activities of obligatory countries. The Kyoto Mechanism consists of Emissions Trading (ET), Clean Development Mechanism (CDM), and Joint Implementation (JI), and among these, Emissions Trading refers to the act of buying and selling carbon emission rights, which are rights to emit greenhouse gas, through the market.

Herein, ‘carbon credit’ is a concept that encompasses allowances and credits, and the quota refer s to the right to emit greenhouse gases paid to major greenhouse gas emission sources such as power generation facilities, production facilities or the like as much as the total amount (cap) of greenhouse gas emission determined within the country or region, and the credit is a certificate that the greenhouse gas emission has been reduced compared to the standard forecast (BAU, Business-As-Usual) for an external greenhouse gas reduction project, and refers to emission rights paid to the corresponding project. Meanwhile, the meaning of the market means that the price of carbon credits is determined by the supply and demand of carbon credits in the market rather than being fixed by policy. This is a way of reflecting the environmental and social costs caused by climate change in the cost of producing goods or services, as opposed to a carbon tax, in which the size of the cost is determined by policy.

Certification according to the amount of carbon reduction used in the present invention a concept corresponding to ‘credit’, and it is a certificate that the greenhouse gas emission has been reduced compared to the standard forecast (BAU, Business-As-Usual) for an externa greenhouse gas reduction project, and may mean having carbon credits paid to the project.

The most representative credit markets are the CDM market and the JI market defined by the Kyoto Protocol. Credit in the CDM market is called Certified Emission Reduction (CER) and credit in the JI market is called Emission Reduction Unit (ER U), and all of these can partially replace quotas such as European Union Allowance (EUA) within quota markers such as EU ETS, and it is possible to reduce the cost burden of the subject of greenhouse gas reduction because the price is usually lower than that of the EUA. Currently, the issuance and transaction volume of CER through the CDM project is overwhelmingly higher than that of ERU through the JI project, and countries around the world are participating in the CDM project. The CDM project is carried out in a format in which a country obligated to reduce in the Kyoto Protocol (Annex I country) invests and develops a greenhouse gas emission reduction project in a developing country (Non-Annex I country). After receiving official certification from an organization that has certified the greenhouse gas emission from the CDM project, carbon credits (CEP) are issued according to the amount of greenhouse gas reduction.

Hereinafter, the method according to various exemplary embodiments of the present invention will be described.

The method according to various exemplary embodiments of the present invention is a method for molecular breeding or providing information of carbon footprint reduction for group subjects. In particular, the method according to various exemplary embodiments of the present invention is a method for improving carbon reduction efficiency using genomic information. This method may be implemented (reflected) based on the following characteristics.

1. Method for Improving Carbon Reduction Efficiency Using Genomic Information (Based on Meat Production)

(1) Breeding to Reduce Carbon Emissions of Livestock Groups

In the case of carbon emission per meat weight (e.g., per kg), it is a quantitative trait that may be quantitatively displayed and shows a continuous distribution. Variations in carbon emissions may be observed in the data of the reference group. In the field of livestock genetic breeding, variation refers to the diversity of subjects as subjects belonging to the same species/group show differences from other subjects other than age, gender or disease, and it may be defined as the difference due to genetic factors to adapt to the environment.

Genetic and environmental factors are acting in a complex way for these variations. In other words, since diversity starts from genetic diversity, it may be used as a fundamental material for animal breeding for variations made by changing genetic material, and it can be said that the diversity of variations represents infinite possibilities of breeding.

These variations exist not only in the natural world, but also may be created artificially, and the development of means and techniques for creating such variations will be with the future and past of livestock breeding. In livestock breeding, the level of quantitative traits of the population is raised by constructing a basic livestock group, discovering and selecting excellent variations that may be used, and pursuing the transfer of the variations to the offspring.
P=G+E
P: Phenotypic Value
G: Genotypic Value
E: Environmental Deviation
G=A+D+I
A: Breeding Value
D: Dominance Effect
I: Epistasis

The reason for the continuous variation of quantitative traits such as carbon emission per meat weight is that these are influenced by a large number of genes, and this type of inheritance is called multi-factor inheritance. In this way, multiple genes are involved when a single trait is expressed, resulting in a cumulative effect.

If the value obtained by measuring a subject's trait is determined by the subject's genotype and environmental factors, the phenotypic value (P) is composed of the genomic value (G) and the environmental deviation (E) when the interaction between genes and environmental factors is not considered. Herein, the genotype value (P) is defined as the sum of the gene effects of all loci that affect a certain trait, and it may be divided into the breeding value corresponding to the additive effect (A), and the dominant effect (D) and epistasis (I) corresponding to non-additive effects.

The breeding value for a quantitative trait that may be quantitatively displayed in a subject is the sum of the effects of the genes that the subject has, and it means the genetic value of the livestock as a parent or seed livestock, and it is a value as a donor who delivers the gene for the next generation. This is a number indicating how much the ability of a subject's offspring differs from the average of the group. Therefore, it is very important to accurately estimate the breeding value of seed livestock or livestock to be used for breeding in order to improve the ability of the entire population.

Meanwhile, it is not easy to correct for various environmental factors such as different breeding farms of each subject, and it is difficult to simultaneously consider the ability of the subject to estimate the breeding value and the ability of all subjects related by blood. However, if the Best Linear Unbiased Prediction (BLUP) proposed by Henderson is used, the ability test data and the pedigree file that may explain the blood relationship between each subject may be used at the same time, and thus, it is possible to estimate the breeding value considering various factors such as the measurer effect and the like. The model, fixed effect, formulas and estimate values for estimating the breeding value are as follows.

$$Y = Xb + Zu + e$$

$$[ \begin{matrix}{X}^{′} X & {X}^{′} Z \\ {Z}^{′} X & {Z}^{′} Z + {A}^{- 1}\end{matrix} ] [ \begin{matrix}\hat{b} \\ \hat{u}\end{matrix} ] = [ \begin{matrix}{X}^{′} X \\ {Z}^{′} X\end{matrix} ] , A = Pedigree λ = \frac{{σ}_{e}^{2}}{{σ}_{a}^{2}}$$

$$[ \begin{matrix}\hat{b} \\ \hat{u}\end{matrix} ] = {[ \begin{matrix}{X}^{′} X & {X}^{′} Z \\ {Z}^{′} X & {Z}^{′} Z + {A}^{- 1} λ\end{matrix} ]}^{- 1} [ \begin{matrix}{X}^{′} & Y \\ {Z}^{′} & Y\end{matrix} ]$$

$$Y : Ability ( observed ) vector$$

$$X : Coefficient matrix for fixed effect b ( enviromental effect )$$

$$Z : Coefficient matrix for breeding value u ( pedigree information )$$

$$b : Fixed effect vec tor / u : Breeding value vector / e : Error vector$$

When estimating the breeding value with this BLUP model, the pedigree relationship matrix is used, and since the breeding value is estimated for each subject included in the lineage, the model used at this time is referred to as an individual model or animal model, and the breeding value for one trait may be estimated (Single Trait Model), or the breeding value for several traits may be estimated at the same time (Multi Trait Model). In general, when the actual breeding value is estimated based on large-scale pedigree records and ability test data, this is called the estimated breeding value. In this case, the data used to estimate and verify the breeding value is called reference data.

That is, the conventional breeding method is efficiently operated at the livestock group level through strategic selection and crossbreeding using breeders related to important economic traits. On the other hand, in the present invention, a breeding method that may be operated in a group is implemented in the direction of increasing the utilization of subjects capable of producing excellent livestock products while reducing carbon by using the carbon emission-related trait as a capability value (breeding value).

(2) Molecular Breeding to Improve the Efficiency of Carbon Emission Reduction Based on Genomic Information

As molecular genetic analysis technology advances, hundreds to millions of SNP markers may be analyzed for one subject. For example, using the Chip technology, it is possible to obtain genotype information for each marker by analyzing about 50,000 SNP markers evenly distributed throughout the genome of cattle, and 50,000 SNP marker genotype information for each subject which undergoes the ability test may be obtained. From the genomics point of view, the effect of each marker on a trait may be calculated by analyzing the relationship between the genotype and the phenotype, and the more subjects with SNP genotype information and phenotype in the reference population, the more accurately the effect of each marker on a trait may be estimated. The effect of each SNP marker may be analyzed using a subject having both such phenotype and marker genotype information. Accordingly, a method for predicting the breeding value of a subject has been developed if there is information on the SNP marker even if there is no phenotype. The breeding value estimated using genomic information in this way is called the genomic estimated breeding value, and this technique is called genomic selection.

By using large-scale reference data, including genomic data, and using thousands to tens of thousands of SNP marker information analyzed for each subject, a genomic relationship matrix (GRM) may be created and a method which is applied to the existing BLUP to simply obtain the breeding value may be used, and the model used in this case is as follows.

$$‐ Using the pedigree relationship matrix ( A ) :$$

$$[ \begin{matrix}\hat{b} \\ \hat{u}\end{matrix} ] = {[ \begin{matrix}{X}^{′} X & {X}^{′} Z \\ {Z}^{′} X & {Z}^{′} Z + {A}^{- 1} λ\end{matrix} ]}^{- 1} [ \begin{matrix}{X}^{′} & Y \\ {Z}^{′} & Y\end{matrix} ]$$

$$‐ Using the genomic relationship matrix ( G ) :$$

$$[ \begin{matrix}\hat{b} \\ \hat{u}\end{matrix} ] = {[ \begin{matrix}{X}^{′} X & {X}^{′} Z \\ {Z}^{′} X & {Z}^{′} Z + {G}^{- 1} λ\end{matrix} ]}^{- 1} [ \begin{matrix}{X}^{′} & Y \\ {Z}^{′} & Y\end{matrix} ]$$

The genomic relationship matrix (G) calculates the degree of having the same genotype between each subject using the SNP marker genotype of each subject. That is, if the pedigree relationship matrix (A) calculates the genetic correlation between each subject based on the pedigree, the genomic relationship matrix (G) calculates the genetic correlation between each subject based on the SNP marker genotype. The genomic relationship matrix (G) may estimate the genetic correlation even when there is no pedigree information, and even if the parents are the same, the SNP marker information for each subject is different, and thus, the genetic correlation is not considered to be identical and the exact genetic correlation may be estimated. The technique of estimating the breeding value of a subject using genomic information may be applied to the genetic evaluation of seed bulls of livestock, and in livestock breeding, it is possible to select subjects by estimating the breeding value with relatively high accuracy using SNP marker information even for various subjects that have not been tested for ability. In particular, when estimating the breeding value of a young subject that has not been tested based on pedigree, offspring born to the same parent are estimated to have the same breeding value. However, if genomic information is used, since the genomic relationship matrix (G) based on the genomic information of each subject is used, the offspring born to the same parent will have different breeding values, and thus, in a livestock group that actively uses seed livestock/artificial insemination, it is possible to secure an accurate breeding value as a whole and select excellent subjects. In the present invention, by constructing reference data by integrating and processing large-scale pedigree information, test-related phenotypic data and genomic data at a level that can be used in the field, and also, by introducing genomic information into the breeding system for reducing carbon emissions of livestock groups, it is possible to implement a molecular breeding method to improve the efficiency of carbon emission reduction that constitutes a selection and high-capacity group with higher accuracy than a breeding system using the existing pedigree information.

(3) Improvement of Efficiency in Reducing Carbon Emission of Korean Cattle Through Molecular Breeding Technology Based on Genomic Information

The fresh weight is information on the weight of live livestock, and the carcass weight is weight information after slaughter, excluding inedible parts such head, viscera, leather and the like. The meat weight refers to the amount of meat that may be obtained after deboning from a carcass, and in general, the amount of carbon emission per meat weight (e.g., per kg) is referred to as the “carbon footprint” of the livestock.

Herein, the carcass percentage is a percentage of the carcass weight in the fresh weight, and the meat percentage is a percentage of the meat weight in the carcass weight. In general, the meat weight is obtained by calculating the meat weight index (meat percentage) from the meat weight information (carcass weight) instead of adding up the amount of meat after deboning for each part, and in the case of Korean beef, the average carcass percentage is about 56% and the average meat percentage is 62%.

In the livestock industry, the carbon footprint per meat weight is generally referred to as the carbon footprint of beef, but since many countries do not manage meat weight information and information on meat mass index, the FAO announces the carbon emission of each country based on carcass weight. However, in the present invention, in the case of Korean beef, since information is systematically managed on subjects that may be identified, the meat weight for calculating the beef carbon footprint may be calculated by multiplying the meat percentage by the carcass weight of the subject.

Meat weight=Carcass weight of subject×Meat percentage

In particular, the amount of carbon emission per meat weight per subject may be proportional to the slaughter age and may be inversely proportional to the meat weight. Therefore, since molecular breeding technology has a predictive function to improve the ability of the group, it is judged that the slaughter age may be controlled in the future, and the meat weight may be set as a target trait to be improved (environmental trait) to reduce carbon emission. For the same resource/time, a subject with a high genetic capacity for meat production will be able to produce more meat than other subjects. That is, since it may reduce the amount of carbon emission per meat weight, it may be estimated that the genetic ability for meat weight is very closely related to carbon reduction.

In addition, conceptually, it is reasonable to define meat weight as a carbon emission-related trait because producing more meat for the input feed means reducing carbon emission and accumulating carbon in the body. In the present invention, meat weight is regarded as a greenhouse gas reduction environmental trait, and through genomic information-based molecular breeding technology, in order to improve carbon emission reduction efficiency at the group level, a genetic resource management system is established for a low-carbon livestock (beef) production system using genome-estimated breeding values with higher accuracy for meat weight.

The breeding value for the meat weight calculated herein may be corrected based on the 900 days (30 months) of slaughtering age because it removes the environmental effect, and accordingly, the slaughter age of the subject in the conversion formula used for carbon emission per meat weight is fixed, and only the meat weight is included in the statistical model for analysis. As a livestock breeder, the meat weight ignores the difference in slaughter age, and thus, it may be differently called as meat production in order to distinguish from the meat weight in the slaughter traceability information.

Y=Xb+Zu+e
Y: Meat weight per subject
X: Environmental effect (year/month of birth, year/month of slaughter, monthly age or yearly age, farm and slaughterhouse, gender)
Z: Pedigree information or genomic information
b: Fixed effect vector/u: Breeding value vector/e: Error vector

The meat weight information is used in the observation vector for the estimation of meat production and the calculation of the breeding price, and as fixed effects on the meat weight, birth year, birth month, slaughter year, slaughter age, age, farm information, slaughterhouse information and gender information are used, and the environmental effect information may be adjusted to improve the accuracy of breeding values. In order to estimate the genetic effect, a relationship matrix is constructed using pedigree and genomes, and the information in the design matrix for this is the genome-estimated breeding value for meat production for improving the carbon reduction efficiency of Korean beef.

(4) Validation of the Effective Amount of Improving the Carbon Reduction Effect of Korean Cattle by Molecular Breeding Technology and the Effect According to the Prediction Accuracy

In livestock breeding, selection (i.e., selection of subjects to be crossed) refers to selecting livestock to be used for breeding in order to produce the next generation from among several subjects in a group, and by selecting genetically superior subjects in the trait to be improved and producing offspring, the genetic composition of the offspring is changed to suit the purpose. In the present invention, an excellent subject is selected and a group is formed efficiently by using the estimated breeding value for the meat weight, and the frequency of a gene or genotype suitable for this purpose is increased, and the frequency of a gene or genotype that is not suitable is reduced or eliminated.

Each subject constituting a livestock group disappears over time, but the group itself is maintained for a long period of time by renewing generations through breeding, and the genetic composition of the next generation is different depending on which subject is selected and leaves offspring in the process of renewing to a new generation through group propagation. If a genetically superior subject is selected and leaves many offspring in the group, the number of subjects with the gene that the subject possessed increases, and as a result, the offspring generation becomes superior to the parent generation, and technically, it is important to increase the efficiency of molecular breeding technology by increasing the prediction accuracy of these excellent subjects.

FIG. 2 shows an example of improving carbon reduction efficiency (based on meat production efficiency) through the input of molecular breeding technology.

According to FIG. 2, the following equations are established.

Group mean of meat weights of the top 50% of the current generation(reference ratio)=Group mean and distribution of meat weights of the next generation(offspring)

Genetic improvement effective amount=Group mean of the top 50% of the current generation(reference ratio)−Total group mean of the current generation

Genetic improvement efficiency=Genetic improvement effective amount/Group mean of the entire meat weights of the current generation

As illustrated in FIG. 2, it is intended to construct the next generation group in the direction of increasing the carbon accumulation rate in the body by using molecular breeding technology, which means improving the ability of the group by selecting excellent subjects for the meat weight. Considering that livestock groups improve their abilities through generations, selecting and using excellent subjects from the current generation for breeding will improve the group mean of the next generation, and in the case of bulls, it is expected that the amount of genetic improvement will be quite high if excellent seed bulls are selected nationwide.

Since the present invention focuses on the possibility of reducing carbon emissions for various groups using molecular breeding technology, when establishing a breeding groupbased on cows, the goal of the group mean and distribution of the next generation is the group at the level of the top 50% (reference ratio) of the current generation, and the effective amount of genetic improvement (the amount of meat increase) is the difference between the group means of the next generation and the current generation, and the actual improvement effect compared to the effective amount of the genetic improvement is related to the prediction accuracy.

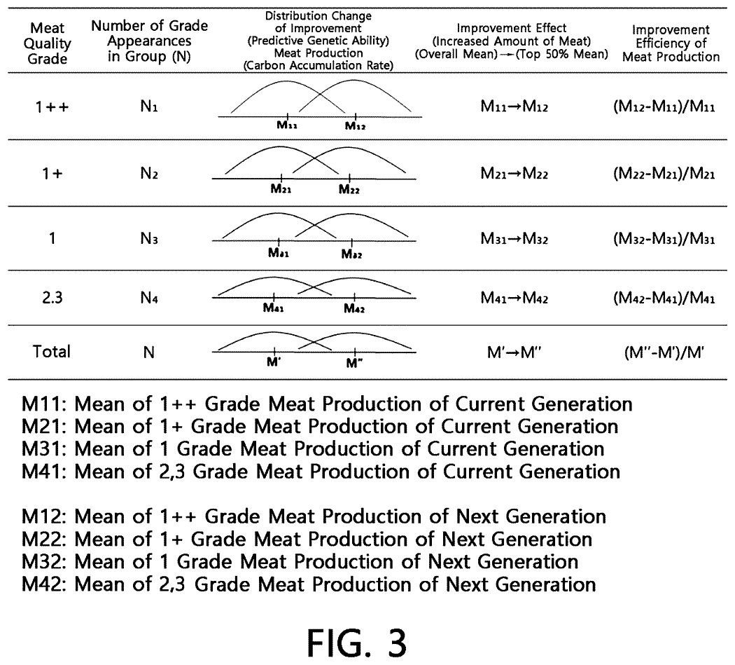

FIG. 3 shows an example of the improvement of carbon emission reduction efficiency based on molecular breeding technology by meat quality grade (based on meat production efficiency).

When the genome-based molecular breeding technology is applied in the actual field, it is essentially to increase the efficiency of the overall beef production system by improving the meat weight, but considering that the current Korean beef market is a meat quality-oriented market, in the present invention, by designing a breeding method in the direction of increasing the meat weight within each meat quality grade, the average value of the group for each grade is increased to increase the efficiency of carbon emission reduction (refer to FIG. 3).

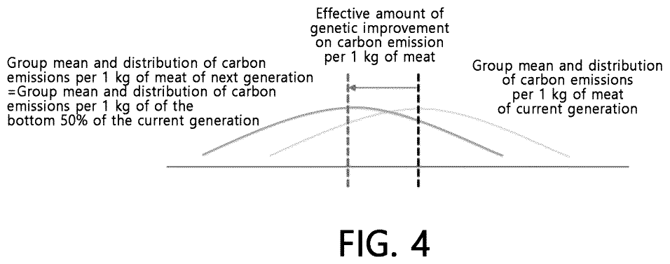

FIG. 4 shows an example of improving carbon reduction efficiency (based on carbon emissions) through the input of molecular breeding technology.

If subjects with excellent meat production are selected and formed into an excellent group, a system capable of producing more meat with the same resources and time may be constructed, thereby reducing carbon emissions per meat weight, and thus, it is possible to reduce carbon emissions per average meat weight at the group level (refer to FIG. 4). Additionally, the formula for the effective amount of genetic improvement and improvement efficiency is the same as that of the meat weight.

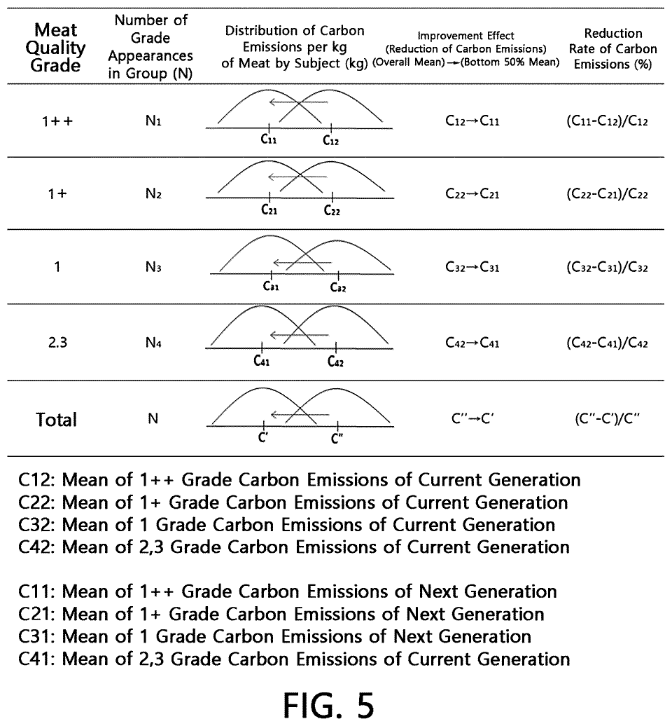

FIG. 5 shows an example of the improvement of carbon reduction efficiency (based on carbon emission) based on molecular breeding technology for each meat quality grade.

Even when calculating carbon emissions by applying molecular breeding technology based on genomic information in the actual field, it is essentially to increase the efficiency of the overall beef production system by improving the meat weight, but considering that the current Korean beef market is a meat quality-oriented market, in the present invention, the efficiency of carbon emission reduction is increased while reducing the mean value of the group for each grade by designing a breeding system in a direction to reduce carbon emissions within each meat quality grade and increase the efficiency therefor. Since the amount of meat production is inversely proportional to the amount of carbon emission per meat weight, it is possible to calculate the amount of carbon emission per meat weight of subjects and calculate the carbon emission reduction amount and the carbon emission reduction rate for each subject (refer to FIG. 5).

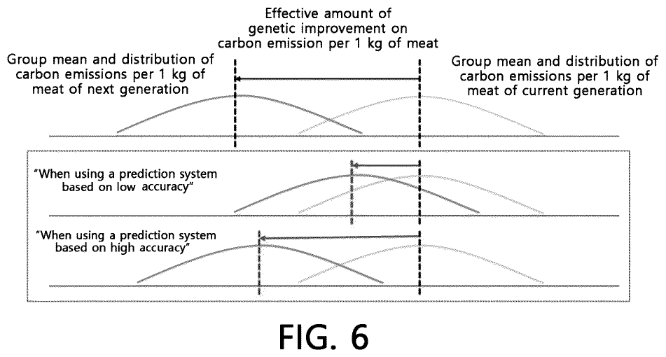

FIG. 6 shows the effect of reducing carbon emissions per meat weight according to the prediction accuracy.

The effective amount of genetic improvement actually implemented in the livestock breeding system is determined by the heritability of the trait and the accuracy of the prediction information (breeding value) used for selection of excellent subjects and group construction, and the heritability of the trait does not change because it is an intrinsic property, but the accuracy of the breeding value may be improved by the data and calculation model used.

The meat weight of Korean cattle is a trait that has almost the same properties as the carcass weight, and the carcass weight is a quantitative trait that is determined by a number of genes, and considering the genome selection study on carcass weight, the predictive accuracy of the breeding value for meat weight is higher when using both pedigree information and genomic information than when using only pedigree information. Based on this, it is judged that the genetic improvement effect related to the emission improvement efficiency is large if a genome-based high-accuracy prediction system is used even in the case of carbon emission per meat weight (refer to FIG. 6).

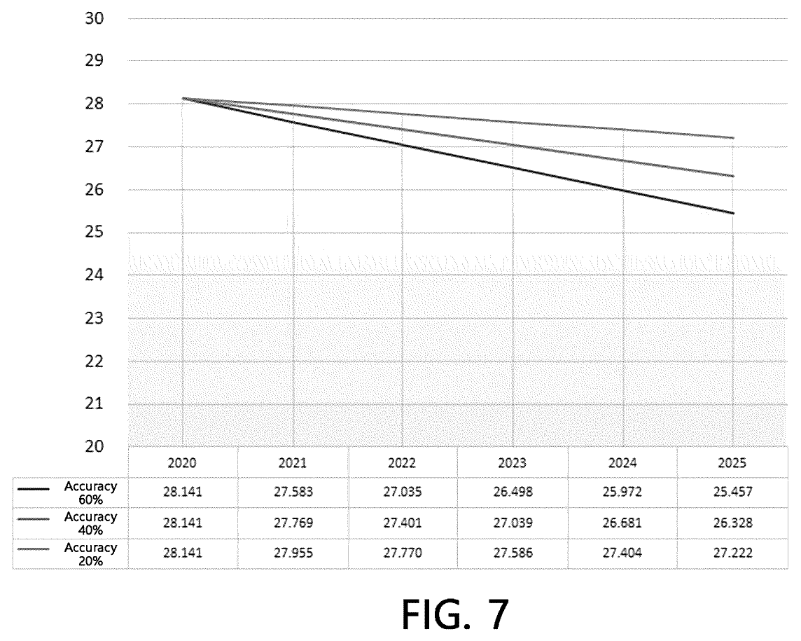

FIG. 7 shows an example of the estimation of the mean of the 1++ grade carbon emission group from 2020 to 2025 by the accuracy of simulation-based breeding values.

If molecular breeding technology is applied based on the amount of carbon emission per meat weight, it is judged that a carbon reduction efficiency improvement system may be established in the direction of reducing carbon emission with a goal of approximately the bottom 50% mean of the overall average. Based on the simulation, the degree of carbon emission reduction per 1 kg of meat per subject may be estimated at the group level based on the heritability (the heritability of 0.37 among the carcass weights of Korean cattle) and the accuracy of the breeding value by 20%, 40% and 60%. The group mean estimates and estimation methods for the accuracy of 20%, 40% and 60% of the breeding value for the carbon emission per meat weight per subject for 5 years in the 1++ grade are as follows. It is confirmed that the higher the accuracy of the breeding value, the greater the reduction in carbon emission (refer to FIG. 7).

2. Case of Demonstrating the Carbon Emission Reduction Effect of the Improvement Group Based on Genetic Ability Prediction

(1) Example of Demonstrating the Effect of Improving the Carbon Accumulation Efficiency (Meat Weight) of the Improvement Group Based on the Genetic Ability Prediction

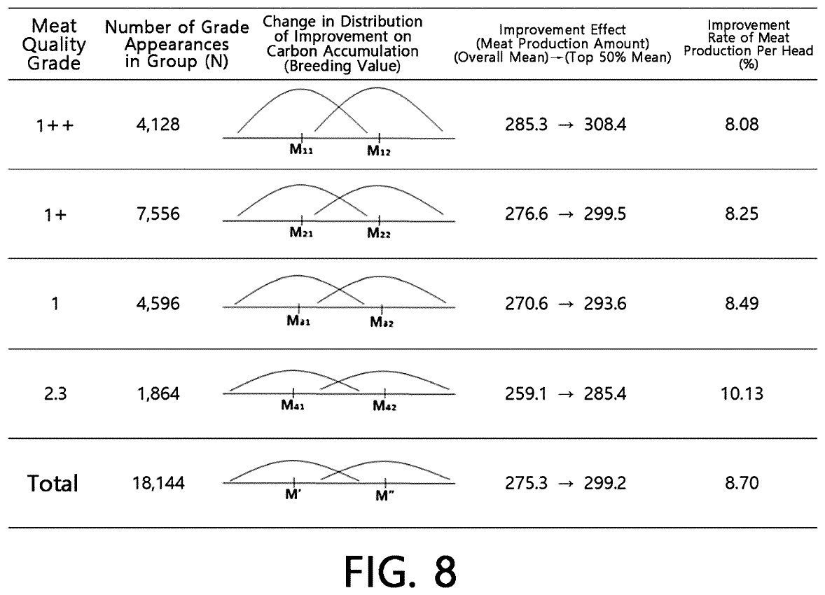

FIG. 8 shows an example of the effect of improving the carbon accumulation efficiency (meat weight) of the improved group after applying the genetic ability prediction.

That is, FIG. 8 shows a case of demonstrating the effect of improving the carbon accumulation efficiency of the improvement group when the genome information utilization technology was applied to a sample group of 18,144 head of Korean beef steer from 2017 to 2019. In this case, the meat was composed of about 21% protein, about 6% fat and about 70% water, and the carbon ratios of protein and fat were about 54.5% and 70%, respectively. That is, when the meat production efficiency increases, the carbon accumulation efficiency increases proportionally. The predicted meat production for each subject was calculated based on the standard meat weight at 30-month-old slaughter, and in the case of selecting the top 50% of the meat production capacity from the whole group and forming the next generation of a steer breeding group, the change in the meat weight (carbon accumulation efficiency) was calculated through predicted empirical results, and it was a reference group with 75% the genetic ability prediction accuracy. The predicted meat production (K) breeding value was calculated based on the amount of information for each subject multiplied by the meat percentage coefficient (0.62) with the carcass weight of the subject. The predicted meat weight is the predicted meat production capacity through the same feed amount at the same slaughter age (30 months of age).

Predicted meat production(K)=Slaughter amount of the top 50% subjects×Meat percentage(0.62)

(2) Case of Demonstrating the Carbon Emission Reduction Effect of the Improvement Group Based on the Prediction of Genetic Ability

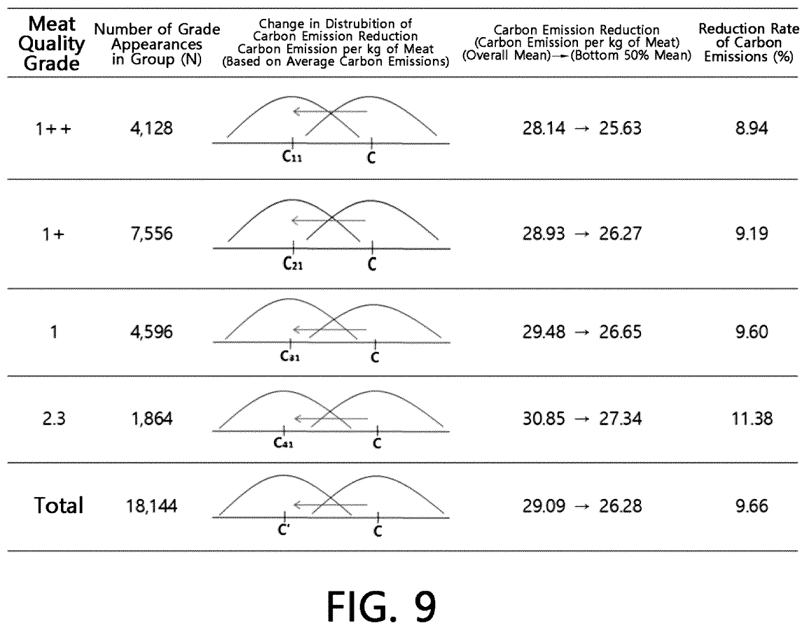

FIG. 9 shows an example of the effect of improving the carbon emission reduction efficiency of the improvement group when the genomic information utilization technology is applied.

The estimated carbon emission per kg of meat for each subject was calculated based on the standard carbon emission for slaughtering at 30 months of age, and when the bottom 50% of carbon emissions was selected from the entire group to form the next generation of a steer breeding group, the change in the carbon emission reduction distribution was calculated through the empirical results of predicting carbon emissions. Carbon emission reduction is calculated using the following equation.

Carbon emission reduction=Group average carbon emission×Average carbon emission reduction rate by subject

The prediction accuracy of 75% is a value calculated based on the predicted value of the meat weight genetic ability (breeding) of a subject using genomic information. The efficiency of meat production improvement or carbon accumulation improvement (%) and the carbon emission reduction effect of selecting the top 50% carbon emission reduction for Korean cattle are calculated using the following equations.

Carbon accumulation improvement efficiency (%)={[(Average of meat weight breeding value for selected group)−(Average for meat weight breeding value for target group)]÷(Average for breeding value for target group)}×100

Carbon emission reduction rate (%)={[(Carbon emission of selected group)−(Carbon emission of target group)]÷(Carbon emission of target group)}×100

The amount of carbon emission reduction through selection of the top 50% of the Korean beef 1++ grade group is calculated using the following equation, and by applying the grade ratio for each grade group in this way, the carbon emission reduction amount for each grade may be calculated.

Carbon emission reduction of Korean beef 1++ grade group=18,144 head×1++ grade ratio×meat weight×28.14 kg (carbon emission per kg of 1++ grade meat)×carbon emission reduction rate

(3) Predicting the Annual Carbon Emission Reduction Effect of Korean Beef Steer Through the Use of the Genetic Ability Prediction Platform

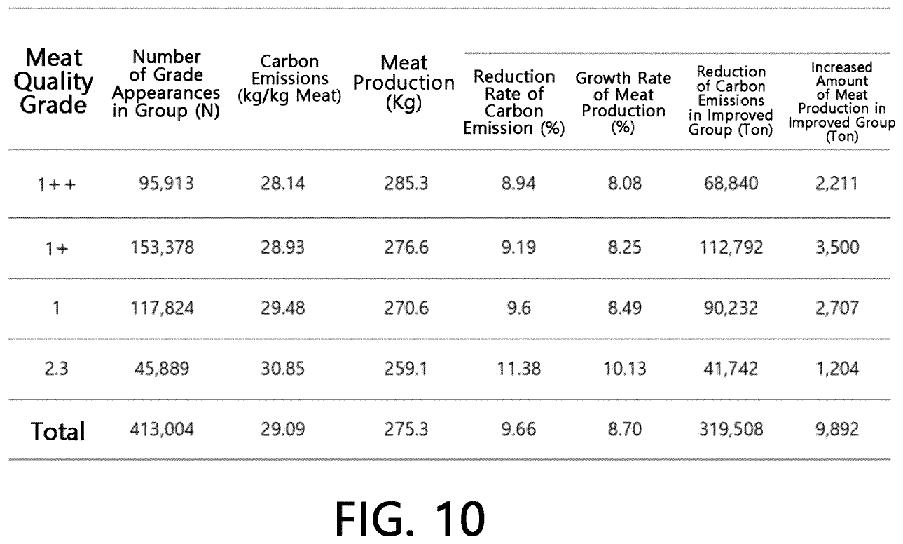

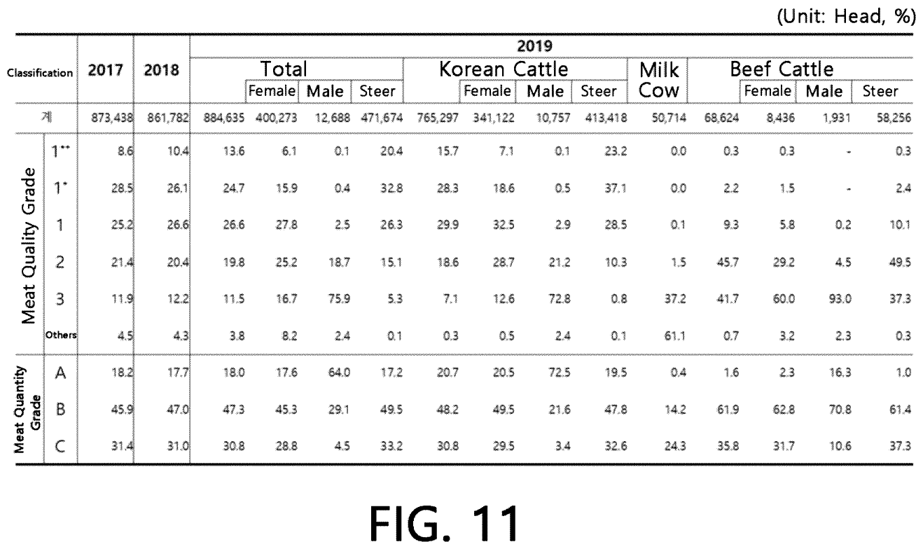

FIG. 10 shows an example of the predicted value of the carbon emission reduction effect of the improvement group (based on the annual production of Korean beef steer) through the application of the genetic ability prediction platform, and FIG. 11 shows the appearance rate by breed, gender, meat quality and meat quantity grades from 2017 to 2019.

That is, FIG. 10 is a prediction result of carbon emission reduction when carbon reduction technology is applied through the use of a genetic ability prediction platform for the 2019 Korean beef steer group (among the slaughter results of 413,418 head of Korean cattle in FIG. 11, data of 413,004 head excluding 0.1% (414 head) of out-of-grade was used). The carbon emission of each predicted subject was calculated based on the meat weight produced during 30 months of breeding, and this is the estimated value of carbon emission reduction calculated based on the meat production standard in the domestic Korean beef steer group, when the top 50% of the meat production capacity was selected from the entire population to produce the next generation of a steer breeding group.

The prediction conditions were that the standard carbon emission per kg of meat was 28.3409 kg, and based on the genetic ability for meat production of the entire steer-producing breeding cattle group, when cows corresponding to the bottom 50% were used for breeding to produce non-beef cattle (introduction of the information utilization and analysis method based on genomic information), the molecular genetic breeding technology for carbon emission reduction (a method of predicting the genetic capacity of meat production and a method of calculating population management and carbon emission reduction therethrough) and the prediction platform were applied.

Under these conditions, 319,508 tons of carbon emissions were reduced every year, and when the total meat production of cows and non-neutered cattle slaughtered past the reproductive age of reproductive cows was taken as the standard (2019: 765,297 slaughtered head), the amount of carbon emission reduction was increased by about 1.85 times and predicted to be 592,049 tons.

In addition to the economic effect by using the genomic information-based meat weight genetic ability and confirming the carbon emission reduction, when adding up the effect of increasing the meat weight according to the present invention, the combined effect may be calculated as the effect of reducing carbon emission+increasing meat production.

Based on the amount of carbon emitted from domestic slaughtered steer (413,004 head), when the present invention is applied, 319,508 tons of carbon emission reduction is expected every year, and if the carbon emission transaction price is calculated at 50,000 won per ton (based on the price of the European carbon credit exchange in April 2021), it is possible to achieve the effect of producing about 16 billion won of carbon credits per year (accumulated amount of carbon credits for 10 years: 160 billion won). In addition to the carbon emission reduction effect of the present invention, the production due to the increase in meat production efficiency is 9,892 tons per year, and when converted to the wholesale market value (33,000 won/kg meat, based on the wholesale transaction price of Korean beef in April 2021), it is expected to be 326.4 billion won per year (the effect of increasing the cumulative meat production over 10 years is 3.264 trillion won).

The 10-year cumulative economic effect on the Korean steer group by this method has the advantage of generating about 3.4 trillion won by adding the carbon reduction effect and the meat production increasing effect. The cumulative economic effect for the entire group of Korean cattle over 10 years is expected to be about 6.3 trillion won, which is multiplied by 1.85 times.

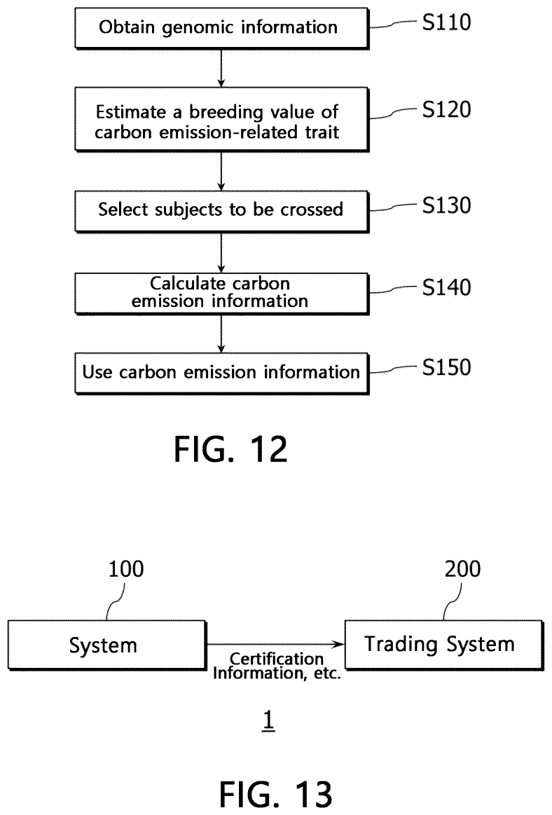

FIG. 12 shows a flowchart of the method according to various exemplary embodiments of the present invention.

The method according to various exemplary embodiments of the present invention may correspond to a method reflecting the above-described characteristics as well as the following contents.

At the national level, for several decades, Korean beef has been improved with a focus on meat weight/meat quality for the production of high-quality beef, quantitatively and qualitatively, and for this purpose, the group's ability has been successfully improved by estimating the breeding value related to slaughtering performance based on the slaughter record and lineage, and selecting excellent seed bulls and cows using the selection index in consideration of the times and market conditions.

In particular, there is a method of estimating the breeding value by constructing a relationship matrix using genome data instead of pedigree using a genome chip that may obtain tens to hundreds of thousands of genome variations (single nucleotide polymorphism; SNP) information. This may predict the ability of subjects more accurately and improve the ability of the group more quickly than breeding values estimated based on the existing lineage, and this method is called genomic selection.

The improvement effect may be brought about by using the genomic selection method for the slaughter performance of Korean cattle, and the genomic selection method is applied to seed bulls and cows. For example, if the carbon emission per weight per subject is used as the standard for carbon emission reduction certification, it is linked to the slaughter age and carcass weight for each subject, and thus, it is possible to improve the carbon emission reduction efficiency by molecular breeding method. If these reduction certification standards are considered, the carbon reduction efficiency of the entire group may be improved by selecting subjects with excellent carcass weight to improve the group's ability regardless of the meat quality grade.

In addition, since it is possible to predict the carcass weight of a subject based on the breeding value, it is possible to adjust the slaughter age for each subject, thereby predicting the carbon emission reduction efficiency. Since this molecular breeding method may predict the subject's ability and increase the carbon emission reduction efficiency in the beef production process, increasing the ability to predict the ability of subjects corresponds to a key technique for improving the efficiency of carbon emission reduction.

When molecular breeding is performed based on genomic information, it is possible to increase the accuracy of the relationship matrix between the reference group (reference subject) and the test group, thereby estimating the breeding value for the slaughter performance, and thereby increasing the efficiency of Korean cattle improvement. That is, in the present invention, by performing the molecular breeding method using the existing data (slaughter/traceability information) and genomic information for the reference group and the genome information for each subject, it is intended to improve the carbon emission reduction efficiency for various groups based on the high predictive degree of carbon emission reduction-related traits for each subject.

As illustrated in FIG. 2, the method according to various exemplary embodiments of the present invention may include S110 to S150.

S110 is a step of obtaining genomic information for each subject of a reference group (i.e., the current generation). For example, the genomic information may include SNPs and the like, but is not limited thereto.

For example, in S110, the corresponding genomic information is received from another device or system through a communicator 120 of a system 100 and stored in a memory 140 of the system 100, or the corresponding genomic information may be transmitted through a removable memory and stored in the memory 140.

Afterwards, S120 is a step of estimating the breeding value of the carbon emission-related trait. That is, in S120, the amount of meat production is set as the carbon emission-related trait of each subject, and then, based on the genomic information obtained in S110, the breeding value of the carbon emission-related trait may be estimated for each subject. In this case, the amount of meat production, which is a carbon emission-related trait, has a characteristic to be inversely proportional to the amount of carbon emission per weight.

For example, in S120, the controller 150 of the system 100 may estimate the breeding value of the meat production by using the genomic information which is pre-stored in the memory 140.

In particular, it is possible to estimate the breeding value by using the following formula.

Y=Xb+Zu+e

In this case, Y is meat production per subject, X is environmental effect information, Z is genomic information, b is a fixed effect vector, u is a breeding value vector, and e is an error vector. In addition, the environmental effect information may be selected from at least one of birth date information, slaughter date information, slaughter age information, farm information, slaughterhouse information and gender information.

The slaughter age information is information on the age of a slaughtered livestock (subject) on the slaughtered day. However, since the corresponding subject is a living subject, the slaughter date information may be fixed (corrected) to 900 days (30 months), and accordingly, the slaughter date information may also be reflected.

Afterwards, S130 is a step of selecting a subject to be crossed. That is, the breeding target subject is a subject selected as a seed livestock (father livestock) or mother livestock because the breeding value of the carbon emission-related trait is excellent among the reference group. In S130, subjects having an excellent breeding value corresponding to a reference ratio or more among the breeding values estimated in S120 may be selected as the breeding target subjects.

For example, in S130, the controller 150 of the system 100 may select subjects having breeding values of meat production corresponding to the top 50% (reference ratio) as the breeding target subjects. However, this reference ratio is not limited to the top 50% and may be applied in various numerical values depending on the situation.

Afterwards, S140 is a step of calculating carbon emission information. That is, in S140, the reference carbon emission information for each subject in the reference group (i.e., the current generation) may be calculated using the breeding value of each subject in the reference group. In addition, in S140, target carbon emission information for each offspring subject (i.e., each subject of the future generation) of the breeding target subjects selected in S130 among the reference group may be calculated using the breeding value of each breeding target subject. However, S140 may be performed before S130 or may be performed together with S130.

For example, in S140, the controller 150 of the system 100 may calculate the above-described reference carbon emission information and target carbon emission information using the breeding value of the corresponding meat production.

In this case, the contents described in the above-described characteristic contents may be used for the method of obtaining the reference carbon emission information and the target carbon emission information and the equations used therefor, and since these contents have already been described above, these will be omitted below.

Afterwards, S150 is a step of using the carbon emission information calculated in S140. For example, in S150, the reference carbon emission information and the target carbon emission information calculated in S140, or a comparison result therefor and the like may be used.

As an example of use in S150, when the reference carbon emission information and the target carbon emission information are compared, the estimated carbon reduction amount is calculated based on the target carbon emission information and the reference carbon emission information, and the estimated carbon reduction amount may be used

In addition, as another example of use in S150, futures trading for carbon credits may be performed based on the estimated carbon reduction amount.

For example, the controller 150 of the system 100 may control the execution of the various examples of use described above. In particular, the controller 150 of the system 100 may access a trading system 200 through a communicator 120 to control the execution of futures trading for carbon credit trading.

In addition, as another example of use in S150, the controller 150 of the system 100 may display the processed information on the display 130 or transmit the same to another device or system. For example, another device or system to be transmitted may be a device used in a farm that breeds at least a part of the reference group, or a system that provides various information to the farm, but is not limited thereto.

For example, when S140 is not performed and S150 is performed, the processed information may include at least one of information about breeding values and information about subjects to be crossed. On the other hand, when S140 and S150 are performed, the processed information may include at least one of information about breeding values, information about subjects to be crossed, reference carbon emission information, target carbon emission information and result information for comparison.

In particular, when the estimated carbon reduction amount is calculated based on the target carbon emission information and the reference carbon emission information, the result information for the comparison may include information on the corresponding estimated carbon reduction amount.

However, in the above-described various examples of use in S150, it is not that only one is performed, and a plurality of examples may be performed together.

In particular, when the controller 150 of the system 100 accesses the trading system 200 to control the carbon credit trading, the system 100 controls to transmit information on the estimated carbon reduction amount to the transaction system 200 through the communicator 120. Certainly, in this case, reference carbon emission information and target carbon emission information may also be transmitted.

Accordingly, the trading system 200 receives and stores information on the estimated carbon reduction amount (or carbon emission amount), and based on the information on the carbon reduction amount (or carbon emission amount), it may control the carbon credit trading between the second and third terminals 20 and 30. Certainly, the trading system 200 may transmit the corresponding information to an intermediary server 40 and the like such that the corresponding carbon credit trading is performed by the mediation of the intermediary server 40.

In addition, for various examples of use in S150, the contents described in the above-described characteristic contents may be used, and since these contents have already been described above, these will be omitted below.

However, the above-described method according to various exemplary embodiments of the present invention may include detailed operations of the above-described systems 1, 100 and 200. However, since the detailed operations have already been described above, these will be omitted below.

The present invention configured as described above proposes a new technique that can specifically derive the predicted carbon reduction amount for offspring subjects (future generation) selected based on the genomic information of the target subjects (current generation), and accordingly, it has an advantage of providing a variety of information related to reducing carbon footprint, molecular breeding and the like. In addition, since the present invention can provide a technique that can calculate and use information on various carbon emissions based on the estimated breeding value by estimating the breeding value of carbon emission-related traits using genomic information, it has an advantage of contributing to greenhouse gas reduction in the livestock sector.

In the detailed description of the present invention, although specific exemplary embodiments have been described, various modifications are possible without departing from the scope of the present invention. Therefore, the scope of the present invention is not limited to the described exemplary embodiments and should be defined by the following claims and their equivalents.

The present invention relates to a method and system for reducing greenhouse gas, and since livestock genomic big data is used to predict the amount of carbon emission for each subject to provide a method and system for reducing the carbon footprint of the corresponding livestock based thereon, it has industrial applicability.

1. A method for molecular breeding for reducing carbon footprint for group subjects, comprising:
obtaining genomic information for each subject in a reference group;
estimating a breeding value of a carbon emission-related trait for each subject based on the genomic information, after setting a meat production amount that is inversely proportional to the amount of carbon emission per weight as the carbon emission-related trait of each subject; and
selecting subjects having a breeding value corresponding to a reference ratio or higher among the breeding values as subjects to be crossed.

2. The method of claim 1, further comprising:
calculating reference carbon emission information, which is information related to the current average carbon emission of each subject, using the breeding value of each subject, and calculating target carbon emission information, which is information related to the estimated average carbon emission amount of each offspring subject of the subjects to be crossed, using the breeding values of the subjects to be crossed; and
using by comparing the reference carbon emission information and the target carbon emission information.

3. The method of claim 2, wherein the using comprises calculating the estimated carbon reduction amount based on the target carbon emission information and the reference carbon emission information to use the estimated carbon reduction amount.

4. The method of claim 3, wherein the using comprises performing futures trading for carbon credit trading based on the estimated carbon reduction amount.

5. The method of claim 1, wherein the estimating comprises estimating the breeding value using the following formula:
Y=Xb+Zu+e
(where Y is the amount of meat production per subject, X is environmental effect information, Z is genomic information, b is a fixed effect vector, u is a breeding value vector, and e is an error vector).

6. The method of claim 5, wherein the environmental effect information is at least one selected from birth date information, slaughter date information, slaughter age information, farm information, slaughterhouse information and gender information.

7. A method performed in a system for providing information for reducing carbon footprint for group subjects, comprising:
storing genomic information for each subject in a reference group;
estimating a breeding value of a carbon emission-related trait for each subject based on the genomic information, after setting a meat production amount that is inversely proportional to the amount of carbon emission per weight as the carbon emission-related trait of each subject;
selecting subjects having a breeding value corresponding to a reference ratio or higher among the breeding values as subjects to be crossed; and
displaying processed information or transmitting the same to another device or system,
wherein the processed information includes at least one of information on the breeding value and information on the subjects to be crossed.

8. The method of claim 7, further comprising:
calculating reference carbon emission information, which is information related to the current average carbon emission of each subject, using the breeding value of each subject, and calculating target carbon emission information, which is information related to the estimated average carbon emission amount of each offspring subject of the subjects to be crossed, using the breeding values of the subjects to be crossed; and
comparing the reference carbon emission information and the target carbon emission information,
wherein the processed information includes at least one of information on the breeding value, information on the subjects to be crossed, the reference carbon emission information, the target carbon emission information and result information from the comparison.

9. The method of claim 8, wherein the comparing comprises calculating the estimated carbon reduction amount based on the target carbon emission information and the reference carbon emission information, and
wherein the result information from the comparison includes information on the estimated carbon reduction amount.

10. The method of claim 9, further comprising performing futures trading for carbon credit trading by accessing a trading system for trading carbon credits based on the information on the estimated carbon reduction amount.

11. A system for providing information for reducing carbon footprint for group subjects, comprising:
a memory for storing genomic information for each subject in a reference group; and
a controller for processing by using the stored information,
wherein the controller estimates a breeding value of a carbon emission-related trait for each subject based on the genomic information, after setting a meat production amount that is inversely proportional to the amount of carbon emission per weight as the carbon emission-related trait of each subject, and selects subjects having a breeding value corresponding to a reference ratio or higher among the breeding values as subjects to be crossed.

12. The system of claim 11, wherein the controller displays processed information on a display or transmits the same to another device of system, and
wherein the processed information includes at least one of information on the breeding value and information on the subjects to be crossed.

13. The system of claim 12, wherein the controller calculates reference carbon emission information, which is information related to the current average carbon emission of each subject, using the breeding value of each subject, calculates target carbon emission information, which is information related to the estimated average carbon emission amount of each offspring subject of the subjects to be crossed, using the breeding values of the subjects to be crossed, and compares the reference carbon emission information and the target carbon emission information,
wherein the processed information includes at least one of information on the breeding value, information on the subjects to be crossed, the reference carbon emission information, the target carbon emission information and result information from the comparison.

14. The system of claim 13, wherein the controller calculates the estimated carbon reduction amount based on the target carbon emission information and the reference carbon emission information upon the comparison, and
wherein the result information from the comparison includes information on the estimated carbon reduction amount.

15. The system of claim 14, wherein the controller performs futures trading for carbon credit trading by accessing a trading system for trading carbon credits based on the information on the estimated carbon reduction amount.
